(19) United States
(12) Patent Application Publication
(10) Pub. No.: US 2003/0023007 A1
(43) Pub. Date: Jan. 30, 2003

(54) Enhancement of thermal properties of benzoxazine polymers by use of aromatic polyamines to incorporate internal benzoxazine groups within the monomer

(75) Inventor: Joseph P. Reardon, Fairview Park, OH (US)

Correspondence Address: D. Peter Hochberg Co., L.P.A., The Baker Building - 6th Floor, 1940 East 6th Street, Cleveland, OH, 44114-2294, US

(73) Assignee: HyComp, Inc.

(21) Appl. No.: 10/077,227

(22) Filed: Feb. 15, 2002

Related U.S. Application Data
(60) Provisional application No. 60/308,217, filed on Jul. 27, 2001.

(51) Int. Cl. C08L 71/02; C07D 265/14

(52) U.S. Cl. 525/430; 544/90

(57) ABSTRACT

A benzoxazine polymer having improved thermal properties. The properties are improved by inserting aromatic polyamines into the monomer, such as phenylenediamine, methylenedianiline, oxydianiline, diaminodiphenylsulfone, 2,2-bis(4-[aminophenoxy]phenyl)propane, 4,4′-oxydianiline, 4,4′-diaminodiphenyl sulfone, and diaminobenzanilide, to introduce internal benzoxazine groups that are crosslinking sites. The improved polymers can be converted into molding compounds, towpregs, and prepregs by being compounded with reinforcing fibers.

### CROSS REFERENCE TO RELATED APPLICATION

This application claims priority of U.S. Provisional Application No. 60/308,217, filed Jul. 27, 2001, under Title 35, United States Code, Section 119(e).

### FIELD OF THE INVENTION

The present invention relates generally to benzoxazine polymers. More particularly, the present invention relates to enhancing the mechanical and thermal properties of benzoxazine polymers via primary aromatic polyamines, which lead to the formation of internal benzoxazine groups within the monomer.

### DESCRIPTION OF THE PRIOR ART

Benzoxazine polymers lend themselves to making many items such as molding compounds, towpregs, and prepregs by being compounded with reinforcing fibers. Benzoxazine polymers are desirable due to their excellent thermal stability and mechanical properties, minimal side reactions, and reasonably fast curing rates. At the same time, benzoxazine polymers have a relatively straightforward chemistry and can be made from reactants that are more economical than other thermoset polymers, such as bismaleimides, polyimides, and cyanate ester resins, that operate in the same desired temperature range as benzoxazine polymers. In comparison, a basic benzoxazine polymer well known in the art and made from two moles of aniline, one mole of bisphenol A, and four moles of formaldehyde has a very reasonable manufacturing cost. However, its glass transition temperature, as measured as the maximum of the loss modulus (E″) curve in differential mechanical thermal analysis, is about only 167° C.-170° C. There is an unsatisfied need to achieve a thermal stability of 200° C. or higher in benzoxazine polymers in a more chemically efficient and cost efficient manner than is presently available with other types of resin.

There have been two basic approaches to constructing benzoxazine monomers that are well known in the art. The first combines monophenol (P) with primary diamine (D) and formaldehyde (F) and having water (H₂O) as a byproduct. It can be represented as 2 P+D+4 F→P-D-P+4 H₂O. Each monomer contains two oxazine rings as the chemical linkages, represented by a “-”. The consumption of two molecules of formaldehyde and the evolution of two molecules of water form each oxazine ring. A second approach combines primary monoamine (A) with diphenol (B) and formaldehyde (F) and having water (H₂O) as a byproduct and can be represented as 2 A+B+4F43 A-B-A+4 H₂O. The second approach is the favored approach in the field. It is important to note that the diamine and diphenol above may also be of higher functionality.

U.S. Pat. No. Re. 32,745 (Schreiber), which embodies earlier Swiss patents, describes a composition having a polymeric resin that is characterized as having particularly outstanding heat stability in combination with good mechanical properties. The composition comprising a polymeric resin is derived from benzoxazine monomers that are co-cured with epoxy resins, in particular with epoxy resins having at least two epoxide groups per molecule, and at least one of the epoxide groups being directly attached to a cyclohexyl ring. The benzoxazine monomers employed are of the type having a monophenol-primary diamine-monophenol stoichiometry or a primary monoamine-diphenol-primary monoamine stoichiometry, but not benzoxazine monomer employing a combination of both diphenols and diamines.

U.S. Pat. No. 4,501,864 (Higginbottom) describes a composition comprising dihydrobenzoxazines and polyamines containing primary or secondary amine groups. The compositions are useful as potting, encapsulating, and laminating resins and as surface coatings.

U.S. Pat. No. 4,507,428 (Higginbottom) describes an aqueous resin dispersion and its use as coating compositions. The aqueous dispersions comprise dihydrobenzoxazine and salts of polyamines.

U.S. Pat. No. 4,557,979 (Higginbottom) describes the deposition of a resin on a metal object, the resin being aqueous dispersions having dihydrobenzoxazine and salts of polyamines.

The three aforementioned Higginbottom patents each present a detailed description of the chemistry of benzoxazine and its pertinence in generating benzoxazine polymers. The patents further describe desirable properties of benzoxazine polymers, such as chemical resistance, toughness, flexibility, and hardness, but do not mention nor discuss thermal stability as a desirable property. Furthermore, Higginbottom describes the processes for synthesizing both monophenol-primary diamine-monophenol benzoxazine monomers and primary monoamine-diphenol-primary monoamine monophenol benzoxazine monomers. It is intended that the two types be mixed after synthesis rather than be synthesized together in one pot.

The patents also describe side reactions during the synthesis of benzoxazine monomers that can adversely affect the properties of the benzoxazine polymers, as well as the criteria for minimizing such undesirable side reactions. The Higginbottom patents emphasize control of the chemical reaction to eliminate the need for subsequent purification steps. The main point of the Higginbottom patents is combining benzoxazine monomer with polyamine co-curative in a stable aqueous dispersion in order to make tough surface coatings and primers.

U.S. Pat. Nos. 5,152,939 and 5,266,695 (Ishida) describe thermally stable benzoxazine polymers and certain reactants needed to achieve them. The patents describe resins with a high char yield for use in making carbon/carbon composites. The patents describe 4,4′-dihydroxybenzophenone, 4,4′-dihydroxybiphenyl, and 1,5′-dihydroxynaphthalene as being excellent reactants for achieving excellent thermal stability when incorporated into benzoxazine polymers. One significant drawback with these reactants is their high cost.

U.S. Pat. No. 5,543,516 (Ishida) describes a solventless system for preparing benzoxazine monomers. Thermal stability is not an issue that is discussed in this patent. The patent teaches that mono-, di-, and tri- functional phenols, as well as mono-, di-, and tri- functional primary amines, are candidate reactants for preparing benzoxazine monomers. The patent teaches that the stoichiometry is well within the skills of those conversant with the art. However, Examples 3, 4, and 5 describe benzoxazine monomers prepared with polyamines and the stoichiometry being such that only simple monomers of the monophenol-primary diamine-monophenol type are produced.

U.S. Pat. No. 5,973,144 (Ishida) describes a benzoxazine polymers having high char yield. The patent discusses that the desired functional groups are those capable of forming chemical crosslinks that are moderately or fully stable to high temperature charring used to form carbon-rich composites. The patent further teaches that the desired functional groups can be an integral part of the amine compound or the phenolic compound or both and that the most thermally stable crosslinks include imides, six membered rings, other cyclic structures, and double bonds derived from acetylene or nitrile.

The patent teaches using three possible mechanisms for increasing char yield: 1) preparing higher molecular weight polymer, 2) forming more temperature stable intermediates, and 3) preventing the volatilization of polymer fragments by increasing their molecular weight or binding them to other polymers. The patent teaches that monophenol-diamine-aldehyde may substitute for monoamine-diphenol-aldehyde; however the employed stoichiometry is intended for monophenol-primary diamine-monophenol and primary monoamine-diphenol-primary monoamine type monomers only, not for higher molecular weight monomers. One drawback to the reactants employed is they are relatively obscure and expensive materials.

U.S. Pat. No. 6,160,079 (Ishida) teaches polybenzoxazines with improved properties due to the use of a substituted arylamine. The patent teaches that the polybenzoxazines of the invention have improved thermal stability between 200° C. and 350° C. The patent teaches the use of an arylamine and stabilizes it by promoting additional chemical bonds to it. This is achieved by substituents, such as alkyl and alkoxy groups, at the meta position of the aryl amine, or at the 5^{th} or 8^{th} carbons of a naphthenic amine, that activate the para position of the phenol for electrophilic aromatic substitution. This results in a more extensive crosslinking, which thus leads to improved thermal stability. The patent teaches that the amine may be a polyamine, although polyamines can yield polyfunctional benzoxazine monomers. However, only monoamines appear in the listed Examples and the employed stoichiometry is for the primary monoamine-diphenol-primary monoamine type of monomer.

### SUMMARY OF THE INVENTION

The present invention is a benzoxazine polymer with enhanced thermal stability and mechanical properties that is derived from a monomer containing internal benzoxazine units in addition to two or more terminal benzoxazine units typical of most such monomers. The internal benzoxazine units are introduced into the monomer by means of aromatic primary polyamines. Because crosslinking occurs at the benzoxazine units, the internal benzoxazine units provide for tighter crosslinking in the polymer, which in turn is manifested as enhanced thermal stability and mechanical properties.

It is an object of the present invention to provide a benzoxazine monomer having an endcap that is an aromatic primary monoamine, a polyphenol, an aldehyde, and an aromatic primary polyamine that serves to introduce internal benzoxazine monomers.

It is another object of the present invention to provide a benzoxazine monomer wherein the aromatic primary monoamine endcap comprises aniline, toluidene, xylidene, or naphthylamine.

It is still another object of the present invention to provide a benzoxazine monomer wherein the polyphenol is a diphenol comprising resorcinol, bisphenol A, bisphenol F (and other isomers thereof), bisphenol S, biphenol, dihydroxybenzophenone, or a triphenol such as trisphenol-PA.

It is still yet another object of the present invention to provide a benzoxazine monomer wherein the aldehyde is formaldehyde comprising any of its various forms, such as paraformaldehyde, trioxane, polyoxymethylene, or formalin.

It is another object of the present invention to provide a benzoxazine monomer wherein the aromatic primary polyamine comprises phenylenediamine, methylenedianiline, oxydianiline, diaminodiphenylsulfone, 2,2-bis(4[4-aminophenoxy]phenyl)propane, or diaminobenzanilide.

It is an object of the present invention to provide a method of preparing such a benzoxazine monomer wherein either a solvent or a slurry is used.

It is also an object of the present invention to provide a polymer wherein a benzoxazine monomer is homopolymerized.

It is another object of the present invention to provide a polymer wherein two or more benzoxazine monomers are blended and cured together.

It is still another object of the present invention to provide a polymer wherein a benzoxazine monomer is co-cured with an epoxy resin.

It is still yet another object of the present invention to provide a polymer wherein a benzoxazine monomer is co-cured with a phenolic resin.

These and other objects of the invention may occur to those skilled in the art from the description to follow and from the appended claims.

### BRIEF DESCRIPTION OF THE DRAWINGS

FIG. 1 is a graph showing the increase in glass transition temperatures as the number of repeat units in benzoxazine monomers in polybenzoxazine made with p-phenylenediamine (PPDA) or 4,4′-methylenedianiline (MDA), compounded with chopped carbon fiber and graphite, and postcured at 400° F., increase.

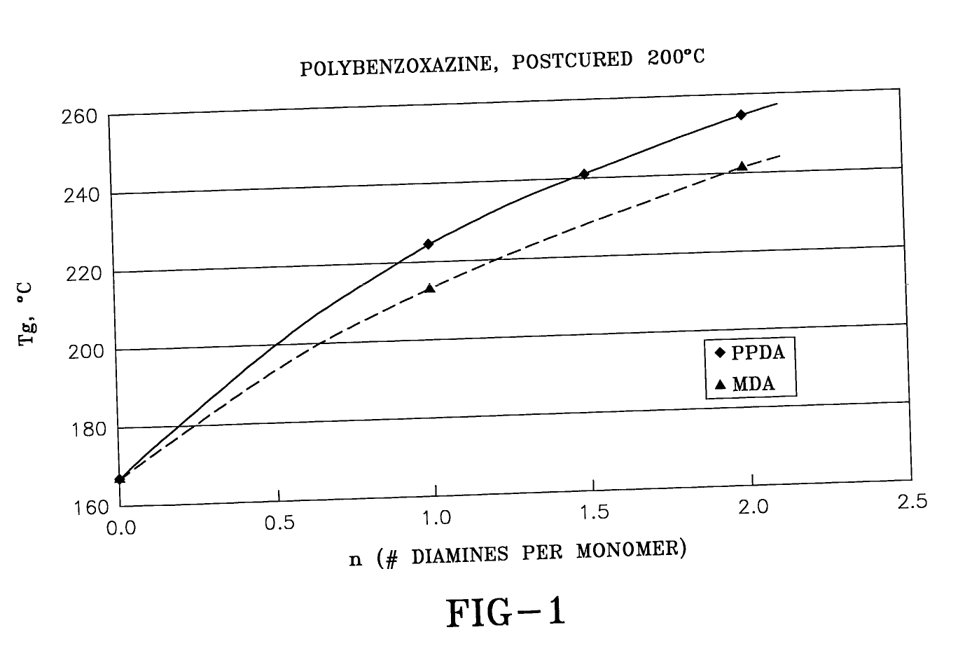

FIG. 2 is a graph showing the increase in glass transition temperature as the corresponding molecular weight of benzoxazine monomers in polybenzoxazine made with PPDA or MDA, compounded with chopped carbon fiber and graphite, and postcured at 400° F. increase.

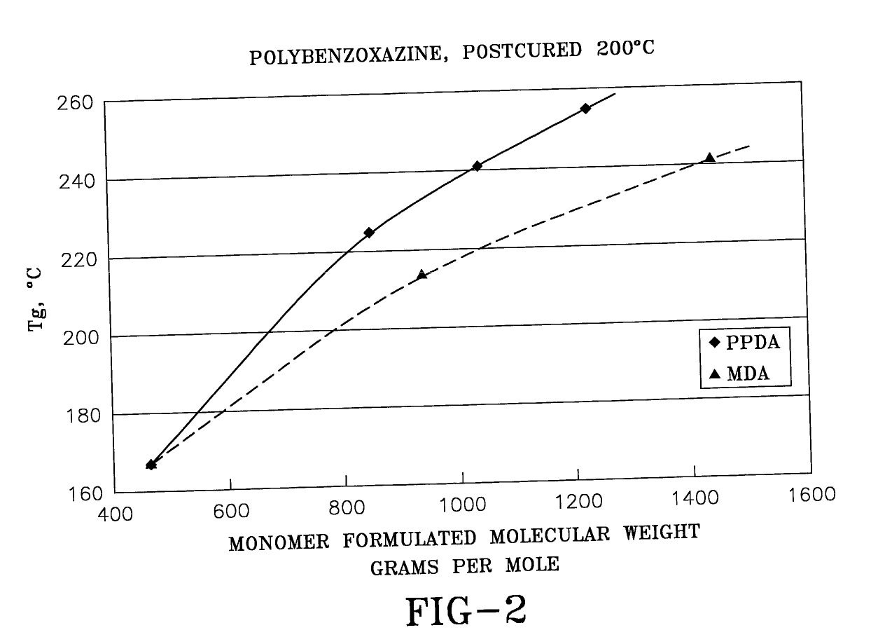

FIG. 3 is a table illustrating the raw data of the polybenzoxazines made with PPDA and MDA and their respective repeating units, molecular weights, and measured glass transition temperatures.

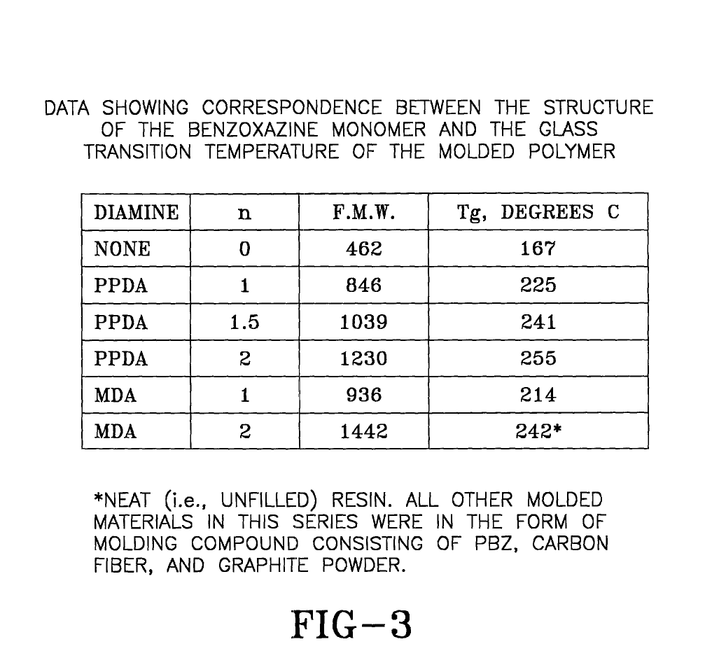

FIG. 4 is a chart showing the structural formulae of aniline, toluidene, xylidene, naphthylamine, phenol, and resorcinol.

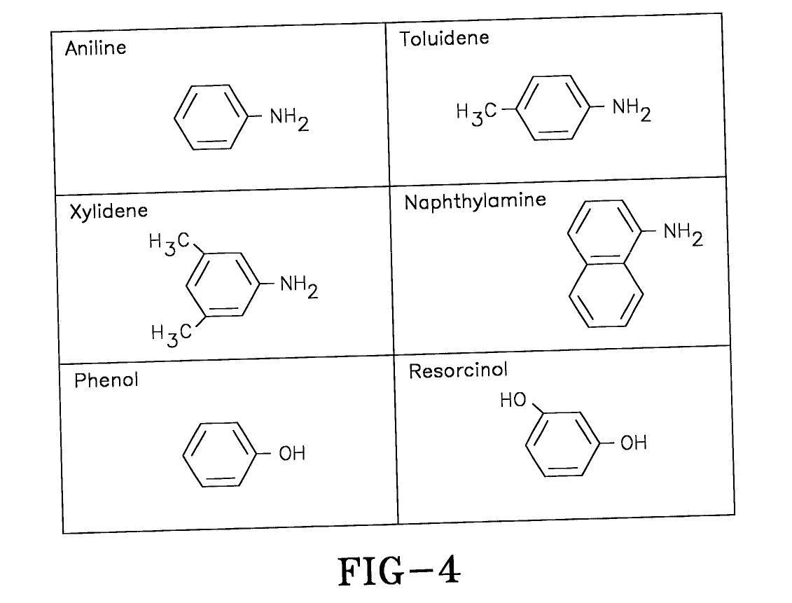

FIG. 5 is a chart showing the structural formulae of p-phenylenediamine, 4,4,′-methylenedianiline, 2,2-bis(4-[4-aminophenoxy]phenyl)propane, 4,4′-oxydianiline, 4,4′-diaminodiphenyl sulfone, and 4,4′-diaminobenzanilide.

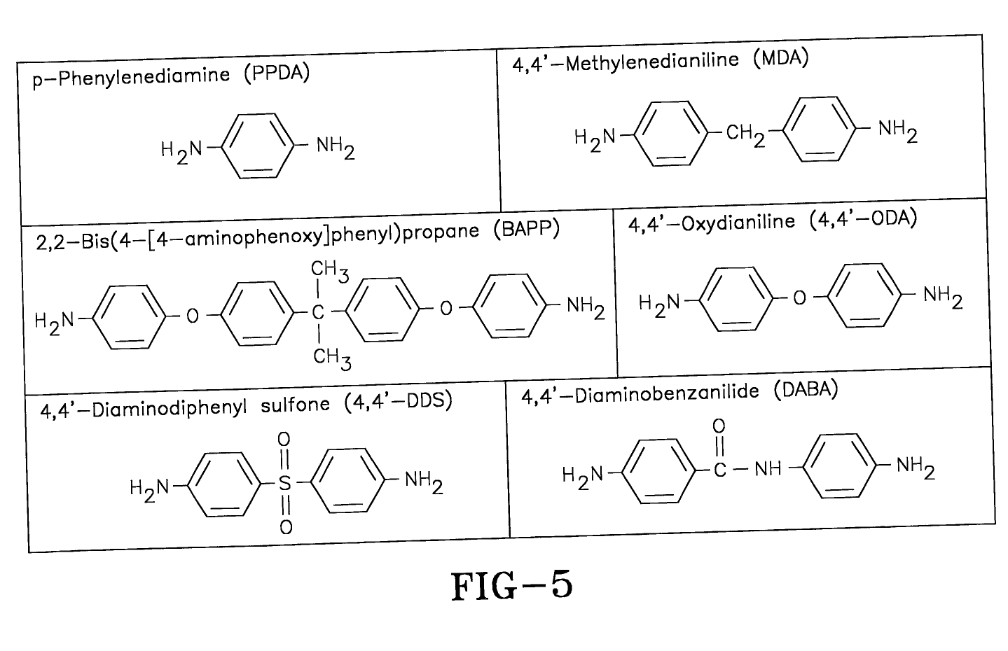

FIG. 6 is a chart showing the structural formulae of bisphenol A, bisphenol F, bisphenol S, biphenol, 4,4′-dihydroxybenzophone, and trisphenol PA.

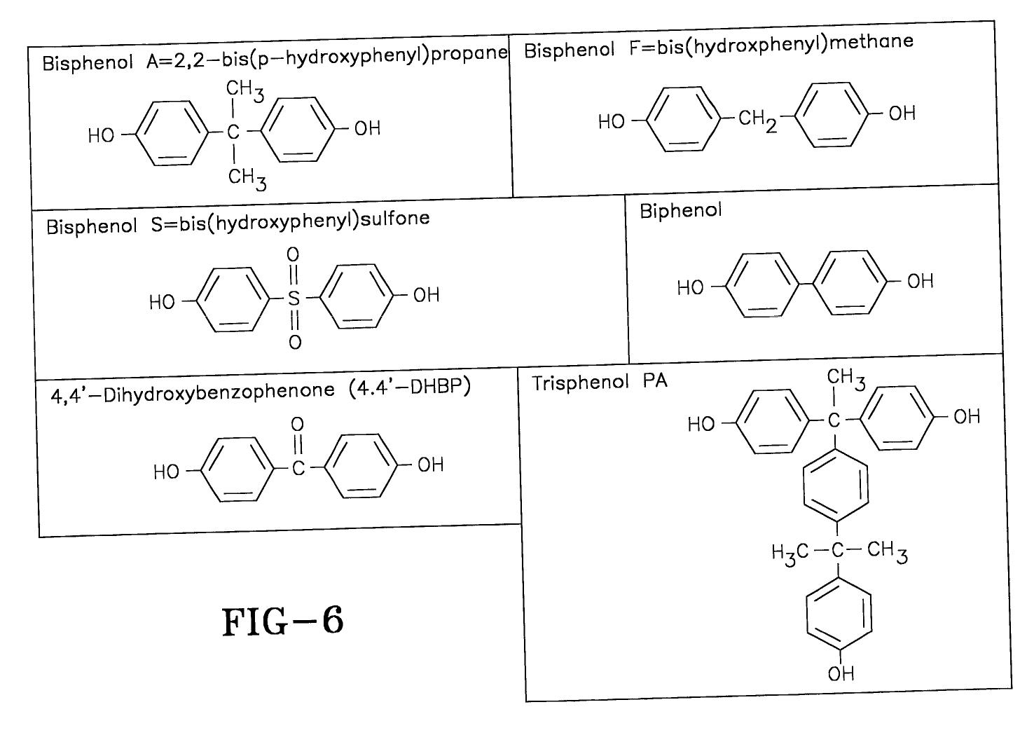

FIG. 7 is a chart showing the structural formulae of formaldehyde, toluene, ethyl acetate, dioxane, methylethyketone, and 1-butanol.

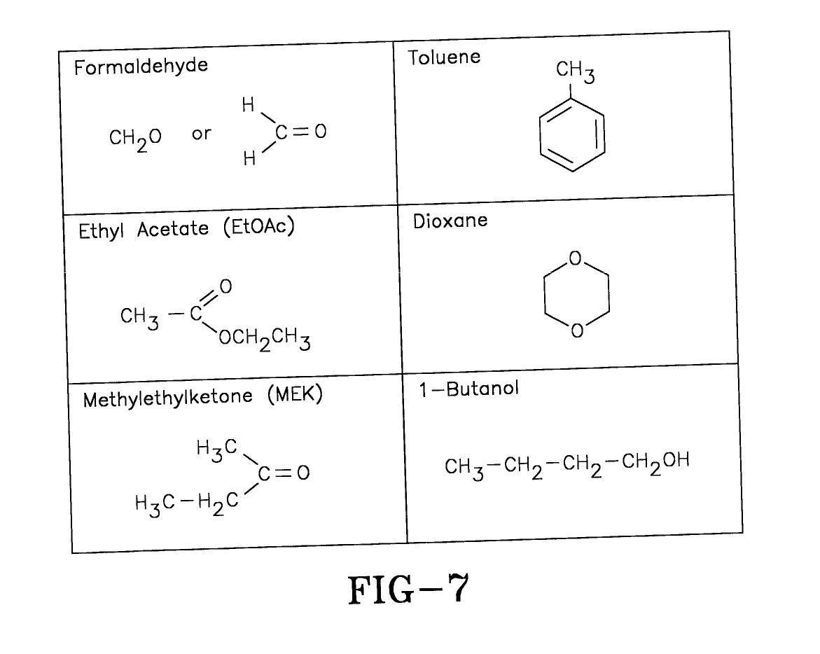

FIG. 8 is a chart showing the structural formula of the polymerization process of the present invention.

### DETAILED DESCRIPTION OF THE INVENTION

Polybenzoxazines, or benzoxazine polymers, having a more economically efficient method of production and having excellent thermal stability due to the use of aromatic polyamines are now described. Polybenzoxazines are desirable polymers because they have reasonably good thermal stability and good mechanical properties at elevated temperatures, while remaining relatively affordable.

Benzoxazine monomers are based on 3, 4 dihydro-1, 3-benzoxazine as shown below:

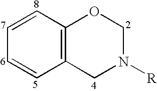

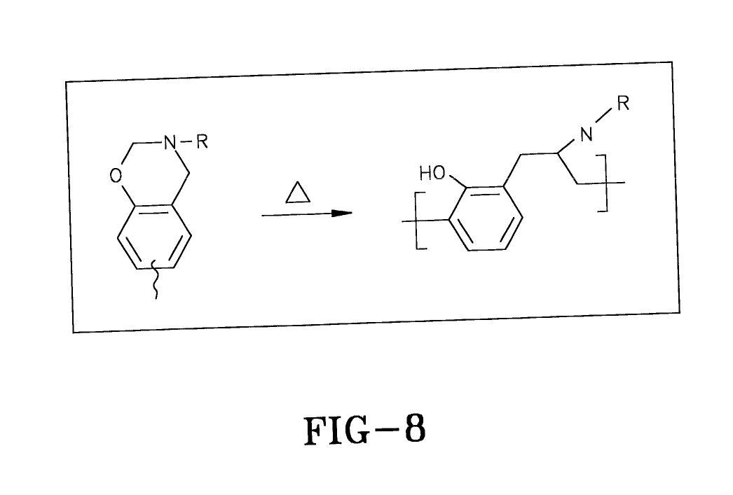

Benzoxazines are known to polymerize via a thermally induced ring-opening reaction to form a phenolic structure that is characterized by a Mannich base bridge. It is believed that the polymerization is an ionic ring opening process which converts the oxazine ring to another structure, for example linear polymer or larger heterocyclic rings. The polymerization process, as depicted in FIG. 8, is shown below:

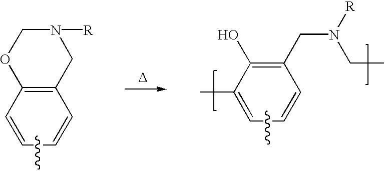

The present invention attains the desired level of thermal stability and a high glass transition temperature (T_{g}) by maximizing the aromatic content of the polybenzoxazines and by increasing the density of chemical crosslinking. Internal benzoxazine rings are able to be introduced by using polyphenols and polyarylamines together in fabricating the benzoxazine monomers.

During the curing step, when the oxazine rings open to polymerize via Mannich bridges, the substituent on the nitrogen in the Mannich bridge of the internal benzoxazine groups is not merely an unconnected pendant group, but rather is an existing molecular chain that contains additional crosslink sites. Thus, an extra degree of potential crosslinking is built into the monomer, in proportion to the number of internal benzoxazine groups, even before the monomer is polymerized. Glass transition temperatures as high as 260° C. confirm this effect.

The benzoxazine monomers used herein are of two types. The first is described as monophenol-(primary diamine-diphenol),-primary diamine-monophenol and the second is described as primary monoamine-(diphenol-primary diamine)ₙ-diphenol-primary monoamine. Hereinafter, the first will be referred to as P-type and the second as A-type and can be represented as P-(D-B)ₙ-D-P and A-(B-D)ₙ-B-A respectively. As stated earlier, each “-” represents an oxazine ring as the chemical linkage. Furthermore, the subscript “n” can equal 0, 1, 2, 3, etc. Because the repeating unit can occur only in integral values, an “n” containing a fractional denotes a mixture. Therefore, “n=1.5” denotes half the monomers would contain one repeating unit, and the other half would contain two.

The present invention employs aromatic primary monoamines or phenols for the endcaps of the benzoxazine monomers. For example, aniline, toluidene, xylidene, or naphthylamine may be employed as aromatic primary monoamines. Of course other isomers of xylidene and naphthylamine may be employed as well. It is noted that any appropriate aromatic primary monoamine may be employed. Examples of phenols to be employed include phenol, cresol, or any other substituted monofunctional phenol. Examples of aromatic primary polyamines to be employed include p-phenylenediamine (PPDA), 4,4′-methylenedianiline (MDA), 4,4′-oxydianiline (ODA), 4,4′-diaminodiphenysulfone (DDS), 4,4′-diaminobenzanilide (DABA), 2,2-bis(4-[4-aminophenoxy]phenyl) propane (BAPP), or 2,4,6-triamino-s-triazine (melamine). Lastly, any appropriate aldehyde may be employed; however formaldehyde, solid paraformaldehyde, or the aqueous/alcoholic solutions of formaldehyde (formalin) are preferred. It is noted that although either aromatic primary monoamines or phenols can be employed for the endcaps of the benzoxazine monomers, all but one of the proceeding examples employ a primary monoamine, namely aniline. This is simply because phenols tend to be more difficult to work with than primary monoamines, although they are interchangeable for purposes of the present invention.

Benzoxazine monomer synthesis may be done in either a solvent or via a solventless method, both of which are methods well known in the art. For purposes of this invention, when the intermediate product is to be chopped fiber molding compound, towpreg, prepreg, and the like, it is preferred that the benzoxazine monomer be prepared via a solvent. When the desired product is to be neat resin powder, then it is preferred that the benzoxazine monomer be prepared via a solventless method.

For benzoxazine monomer syntheses made in solvent, it is preferred that a mixture of toluene and ethyl acetate be employed. It is preferred that the ratio of toluene/ethyl acetate be about 8:1 to about 6:1. However, any combination of the aforementioned solvents from about 10:1 to about 4:1 may be employed. Using a combination of solvents is advantageous in that the collection of water by azeotropic distillation is expedited. Contrastly, employing either toluene or ethyl acetate alone results in a slow and tedious distillation process. The combination of solvents is also advantageous in that it allows the separation of water from solvent in a Barrett or Dean-Stark distillation trap to be sharp and allowing nearly all the solvent to be returned to the reaction kettle.

Employing formalin in addition to paraformaldehyde in preparing the benzoxazine monomer is also advantageous. Paraformaldehyde is preferred as it is significantly less expensive than formalin. The solid paraformaldehyde, however, is insoluble in nearly all organic solvents. This forces the reaction to occur at the solid-liquid interface, thus significantly limiting the rate of reaction. Employing formalin in combination with the paraformaldehyde provides enough water and methanol to dissolve the paraformaldehyde. Alternatively, just water may be used. Formalin is also advantageous in that it mitigates the exotherm that occurs at about 80° C.-85° C. A violent exotherm occurs because as water is generated more paraformaldehyde can dissolve, thus rapidly accelerating the reaction rate.

On the contrary, with benzoxazine monomer preparation in solvent, although all the formaldehyde may be provided as formalin, this is an undesirable method because formalin is expensive and it introduces an unnecessary amount of water into the system which must be removed later. It is advantageous to employ a paraformaldehyde/formalin ratio of at least 1:1, based on the dry weight of the formaldehyde, and preferably of about 5:1.

Preparation of Benzoxazine Monomers in Solvent

A reaction kettle is kept at no warmer than about 25° C. while ingredients are added. Alternatively, reaction kettle may be cooled with ice, or any other cooling mechanism. Solvents, such as toluene and ethyl acetate, are added to the reaction kettle. Next, liquid reactants such as formalin or aniline are added. Then, the solids such as PPDA flakes, paraformaldehyde granules, or bisphenol A powder are slowly added to the mixture so as to avoid clumping. Each reactant is added to the kettle in an alternating fashion, adding only a small portion of the entire amount at once. Each reactant is added until the fall amounts have been added to the kettle. This is done so as to avoid any undesirable adverse reactions that could potentially occur by adding the reactants as a whole. This mixture is slowly warmed to about 82° C.-85° C. at which point an exothermic reaction in the form of rapid boiling occurs. The kettle is maintained in reflux for about 1-2 hours and the water byproduct is collected by any method conventional in the art, such as via a Barrett trap.

Once it is deemed that the reaction is sufficiently advanced, the resinous liquid is poured from the kettle. This is a visual determination that is reached once the material in the kettle becomes a light colored, homogenous creamy liquid with no visible particulates. The liquid is removed from the kettle so as to be mixed into a molding compound, impregnate onto fabric, or the like. The molding compounds of the proceeding examples have the composition by weight of about 10% graphite powder, 35% milled carbon filler, and 55% polybenzoxazine, as cured. It is appreciated that other compositions with other fillers that are known in the art can also be used.

As the liquid is allowed to cool to about 30° C., it becomes “snotty” or “stringy” indicating the presence of resinous benzoxazine. Once the molding compound has been mixed or fabric impregnated, etc., the “wet” product is dried by any means conventional in the art. Preferably, the wet product is dried first in a vacuum oven at about 65° C. to about 70° C. This step sufficiently removes any remaining solvent and water. The product is then transferred to an air-circulating oven where it is taken to its melt temperature, which is at about 100° C. to about 130° C. The product is held at this temperature for about 30 minutes to about 4 hours so as to complete the reaction and to form the final benzoxazine monomers.

The neat resin or molding compound can then be ground or granulated by any means conventional in the art, such as by ballmilling. Alternatively, the product can be ground down by hand.

The product is now ready to be tested. The ground product is compression-molded into test plaques. The test plaques generally have dimensions of about 9×13 ×0.64 cm³ or 28×41×0.64 cm³. The test plaques are cured at about 215° C.-230°C. and 3.5 Mpa for about 20 minutes and machined into bars for subsequent conditioning and testing. The tested bars may be conditioned via post-curing in an oven at about 204° C.-230° C.

The test bars are measured for density. The bars are then tested in flexure at any desirable temperature between ambient and 177° C. so as to determine the strength and modulus of each specimen. Finally, selected bars are tested for glass transition temperature via thermal analysis, preferably differential mechanical thermal analysis (DMA).

Production of Resin Powder Via a Slurry

Production of resin powder as the final product can be performed employing conventional methods. However, introducing an organic solvent into the benzoxazine monomer synthesis serves no useful purpose. A solventless process, although applicable here, has practical problems such as difficulty in thoroughly mixing the reactants, especially because solids predominate, and a violent exothermic reaction.

It is advantageous to combine reactants in the reaction kettle as an aqueous/alcoholic slurry. Formalin is employed as the sole, or substantial, source of formaldehyde. Formalin supplies enough water, or water/methanol, to make an easily stirred slurry of the other reactants. It is also advantageous to combine the reactants for mixing at or about 30° C. or less, and slowly warm them with vigorous stirring. The slurry will become smooth and homogenous at about 60° C. to about 65° C. The exothermic reaction occurs at about 85° C. and lasts for a few minutes and the resin subsequently turns into a taffy-like formation. The water (or water-methanol) byproduct separates and is poured off. The resulting molten monomer is poured from the reaction kettle for additional processing.

### EXAMPLES

### Example 1

Benzoxazine Resin Without Polyamine (n=0) Prepared in Solvent; F.M.W.=462 g/mole

A 3000-mL glass kettle was placed in an ice bath to keep it cool. 600 g of toluene and 100 g of ethyl acetate were added, followed by 168.5 g of formalin (2.08 moles), 483.2 g of aniline (5.19 moles), 262.4 g. of paraformaldehyde (8.30 moles), and 592.3 g of bisphenol A (2.60 moles). The mixture was slowly heated to reflux at 84C. Reflux was maintained for 3½ hr., during which time 184 g of aqueous phase was removed (out of calculated 326 g total). The final resin solution was off-white in color and opaque, with no visible particulates.

The resin solution was converted into molding compound by mixing 1100 g of it with 471.4 g of milled carbon fiber and 134.6 g of graphite powder. The wet mixture was first dried in a vacuum oven at about 70C for 1½ hr and then in an air-circulating oven at 110C for 3 hr more. The dried molding compound was granulated and then molded into a plaque measuring 41 cm×28 cm×0.58 cm, with a density of 4.50 g/cc. Flexural test bars were machined from the plaque and tested. Flexural strength averaged 81.9 MPa for as-molded test bars and 77.4 MPa for postcured bars (204C for 6 hr). Flexural moduli were 16.0 GPa and 16.7 GPa respectively. Glass transition temperature by DMA (E″ max.) was 167C.

### Example 2

Benzoxazine Resin with PPDA (n=1) Prepared in Solvent; F.M.W.=846 g/mole

A 3000-mL glass kettle was placed in an ice bath to keep it cool. 700 g of toluene and 100 g of ethyl acetate were added, followed by 230.0 g of formalin (2.84 moles), 264.0 g of aniline (2.84 moles), 268.8 g of paraformaldehyde (8.51 moles), 153.2 g of p-phenylenediamine (1.42 moles), and 646.8 g of bisphenol A (2.84 moles). The mixture was slowly heated to reflux at 83C. Reflux was maintained for about 2 hr, during which time 152 g of aqueous phase was removed (out of calculated 386 g total). The final resin solution was tan, opaque and snotty, but it poured well.

The resin solution was converted into molding compound by mixing 1220 g of it with 414.6 g of milled carbon fiber and 118.5 g of graphite powder. The wet mixture was first dried in a vacuum oven at about 70C for ¾ hr and then in an air-circulating oven at 113C for 3 hr more. The dried molding compound was granulated and then molded into a plaque measuring 41 cm×28 cm×0.65 cm, with a density of 1.42 g/cc. Flexural test bars were machined from the plaque and tested. Flexural strength averaged 80.9 MPa for as-molded test bars and 81.2 MPa for postcured bars (204C for 6 hr). Flexural moduli were 15.9 GPa and 15.4 GPa respectively. Glass transition temperature by DMA (E″ max.) was 225C.

### Example 3

Benzoxazine Resin with PPDA (n=1.5) Prepared in Solvent; F.M.W.=1039 g/mole

A 3000-mL glass kettle was placed in an ice bath to keep it cool. 770 g of toluene and 130 g of ethyl acetate were added, followed by 210.8 g of formalin (2.60 moles), 242 g of aniline (2.60 moles), 328.4 g of paraformaldehyde (10.39 moles), 210.7 g of PPDA (1.95 moles), and 741.3 g of bisphenol A (3.25 moles). The mixture was slowly heated to reflux at 83° C. Reflux was maintained for about 2 hr, during which time 145 g of aqueous phase was removed (out of calculated 398 g total). The final resin solution had a uniform tan color and was relatively fluid. The resin solution was converted into molding compound by mixing 1620 g of it with 561.3 g of milled carbon fiber and 160.5 g of graphite powder. The wet mixture was first dried in a vacuum oven at about 70C for 1.5 hr and then in an air-circulating oven at 113° C. for 3 hr more. The dried molding compound was granulated and then molded into a plaque measuring 9 cm×13 cm×0.70 cm, with a density of 1.387 g/cc. Flexural test bars were machined from the plaque and tested. Flexural strength averaged 92.6 MPa for as-molded test bars. Flexural modulus was 15.6 GPa. Glass transition temperature by DMA (E″ max.) was 241° C.

### Example 4

Benzoxazine Resin with PPDA (n=2) Prepared in Toluene; F.M.W.=1230 g/mole

A 1000-mL glass kettle was placed in an ice bath to keep it cool. 200 g of toluene were added, followed by 45.91 g of aniline (0.49 moles), 93.33 g of paraformaldehyde (2.95 moles), 52.75 g of PPDA (0.49 moles), and 166.8 g of bisphenol A (0.73 moles). The mixture was slowly heated to reflux at 84° C. Reflux was maintained for about 10 min, no aqueous phase was removed. The final resin solution was dark brown and contained part slurry and part solubilized resin.

The resin solution was converted into molding compound by mixing it (558 g) with 190.9 g of milled carbon fiber and 54.5 g of graphite powder and 100 g of toluene. The wet mixture was dried in an air-circulating oven at 113° C. for 3 hr. The dried molding compound was granulated and then molded into a plaque measuring 9 cm×13 cm×0.82 cm, with a density of 1.404 g/cc. Flexural test bars were machined from the plaque and tested. Flexural strength averaged 79.6 MPa for as-molded test. Flexural modulus was 18.2 GPa. Glass transition temperature by DMA (E″ max.) was 25520 C.

### Example 5

Benzoxazine Resin with PPDA (n=2) Prepared in Ethylacetate; F.M.W. =1230 g/mole

A 1 000-mL glass kettle was placed in an ice bath to keep it cool. 200 g of ethyl acetate were added, followed by 45.9 g of aniline (0.49 moles), 93.4 g of paraformaldehyde (2.96 moles), 52.8 g of PPDA (0.49 moles), and 166.9 g of bisphenol A (0.73 moles). The mixture was slowly heated to reflux at 84° C. Reflux was maintained for about 30 min, during which time 2.4 g of aqueous phase was removed (out of calculated 58 g total). The final resin solution was caramel tan color with white particulates of paraform within.

The resin solution was converted into molding compound by mixing it (556 g) with 190.8 g of milled carbon fiber and 54.5 g of graphite powder and 50 grams of ethyl acetate. The wet mixture was first dried in an air-circulating oven at 104° C. for 4 hr. The dried molding compound was granulated and then molded into a plaque measuring 9 cm×13 cm×0.60 cm, with a density of 1.395 g/cc. Flexural test bars were machined from the plaque and tested. Flexural strength averaged 109.7 MPa for as-molded test bars. Flexural modulus was 22.5 GPa. Glass transition temperature by DMA (E″ max.) was 260° C. when postcured at 400° C. and 254° C. as-molded.

### Example 6

Benzoxazine Resin with MDA (n=1) Prepared in Aqueous Slurry: F.M.W.=936 g/mole

A 1000-mL glass kettle was placed in an ice bath to keep it cool. Added to the kettle were 155.9 g of formalin (1.92 moles), 44.7 g of aniline (0.23 mole), 47.6 g of 4,4′-methylenedianiline (0.23 mole), and 109.6 g of bisphenol A (0.480 mole). The kettle was heated slowly. At 63C the stirrer was removed. Shortly after a mild exotherm at 85C, the material turned taffy-like. It was held at 88-93C for 20 min. to promote full reaction. The final product was a tan material with putty consistency, with a thin layer of water covering the top.

The material was dried in an air-circulating oven for 2-⅓ hr at 113C. The dried “cake” was broken into small chunks and ballmilled into a fine, off-white powder. Some of the powder was molded into a plaque 9 cm×13 cm×0.71 cm, with a density of 1.18 g/cc. Flexural test bars were machined from the plaque and tested. Flexural strength averaged 78.6 MPa for the as-molded test bars. Flexural modulus was 6.4 GPa. Glass transition temperature by DMA (E″ max.) was 207C.

### Example 7

Benzoxazine Resin with MDA (n=1) Prepared in Solvent: F.M.W.=936 g/mole

A 3000-mL glass kettle was placed in an ice bath to keep it cool. 700 g of toluene and 100 g of ethyl acetate were added, followed by 187.1 g of formalin (2.31 moles), 268.2 g of aniline (2.88 moles), 291.5 g of paraformaldehyde (9.22 moles), 285.5 g of MDA (1.44 moles), and 657.7 g of bisphenol A (2.88 moles). The mixture was slowly heated to reflux at 84° C. Reflux was maintained for about 4 hrs, during which time 184g of aqueous phase was removed (out of calculated 372 g total). The final resin solution was creamy white and slightly snotty.

The resin solution was converted into molding compound by mixing 1155.7 g of it with 429.5 g of milled carbon fiber and 122.7 g of graphite powder. The wet mixture was first dried in a vacuum oven at about 70° C. for 1.5 hr and then in an air-circulating oven at 1 02° C. for 2 hr more. The dried molding compound was granulated and then molded into a plaque measuring 28 cm×41 cm×0.52 cm, with a density of 1.425 g/cc. Flexural test bars were machined from the plaque and tested. Flexural strength averaged 100 MPa for as-molded test bars and 99.9 MPa for postcured bars (400° C. for 6 hr). Flexural moduli were 17.4 GPa and 16.7 GPa respectively. Glass transition temperature by DMA (E″ max.) was 214° C.

### Example 8

Benzoxazine Resin with MDA (n=2) Prepared in Solvent; F.M.W. 1442 g/mole

A 1000-mL glass kettle was placed in an ice bath to keep it cool. 170 g of toluene and 30 g of ethyl acetate were added, followed by 41.4 g of formalin (0.51 moles), 39.6 g of aniline (0.43 moles), 64.5 g of paraformaldehyde (2.04 moles), 84.3 g of MDA (0.43 moles), and 145.5 g of bisphenol A (0.64 moles). The mixture was slowly heated to reflux at 84° C. Reflux was maintained for about 2 hr, during which time 64 g of aqueous phase was removed (out of calculated 82.4 g total). The final resin solution was off-white in color and extremely viscous but pourable when hot. At room temperature, it had a consistency similar to petroleum jelly.

The resin was first dried in a vacuum oven at about 70° C. for 1 ¼ hr and then in an air-circulating oven at 113° C. for 2 ⅓ hr more. When dried, the resin (about 300 g) was milled into a fine powder in a 1.8-L alumina jarmill. Next, 75 g of graphite powder were added. Tumbling for 20 minutes gave a homogeneous blend of the two powders. This molding compound was then molded into a plaque measuring 9 cm×13 cm×0.68 cm, with a density of 1.305 g/cc. Flexural test bars were machined from the plaque and tested. Flexural strength averaged 44.3 MPa for as-molded test bars. Flexural modulus was 8.4 GPa. Glass transition temperature by DMA (E″ max.) was 242° C.

### Example 9

Benzoxazine Resin with MDA (n=3) Prepared in Solvent; F.M.W. 1884 g/mole

A 1000-mL glass kettle was placed in an ice bath to keep it cool. 170.1 g of toluene and 40.5 g of ethyl acetate were added, followed by 43.5 g of formalin (0.54 moles), 31.1 g of aniline (0.33 moles), 67.4 g of paraformaldehyde (2.13 moles), 99.2 g of MDA (0.50 moles), and 152.3 g of bisphenol A (0.67 moles). The mixture was slowly heated to reflux at 83° C. Reflux was maintained for about 1 hr, during which time 47 g of aqueous phase was removed (out of calculated 82.4 g total). The final resin solution was coagulated into a rubbery, non-tacky glob.

The mixture was dried in an air-circulating oven at 113° C. for 7 hr. The dried molding compound was granulated and then molded into a plaque measuring 9 cm×13 cm×0.68 cm, with a density of 1.169 g/cc. Flexural test bars were machined from the plaque and tested. Flexural strength averaged 76.8 MPa for as-molded test bars. Flexural modulus was 5.3 GPa.

### Example 10

Benzoxazine Resin with BAPP (n=1) Prepared in Solvent; F.M.W.=1149 g/mole

A 1000-mL glass kettle was placed in an ice bath to keep it cool. 166 g of toluene and 40 g of ethyl acetate were added, followed by 34.7 g of formalin (0.43 moles), 48.6 g of aniline (0.52 moles), 52.8 g of paraformaldehyde (1.67 moles), 107.8 g of 2, 2-bis (4-[4-aminophenoxy]phenyl) propane (BAPP) (0.26 moles), and 119.3 g of bisphenol A (0.52 moles). The mixture was slowly heated to reflux at 84° C. Reflux was maintained for about 1 and two-thirds hr, during which time 53.6 g of aqueous phase was removed (out of calculated 67.5 g total). The final resin solution was fluid and mostly clear.

The resin was dried in an air-circulating oven at 113° C. for 5 hr. The dried resin was broken into small chunks and then molded into a plaque measuring 9 cm×13 cm×0.64 cm, with a density of 1.163 g/cc. Flexural test bars were machined from the plaque and tested. Flexural strength averaged 84.7 MPa for as-molded test bars. Flexural modulus was 4.3 GPa.

### Example 11

Benzoxazine Resin From an Equimolar Blend of Monomers with PPDA (n =1) and BAPP (n=1)

Portions of the monomer solutions of Example 2 (511 g) and Example 10 (649 g) were mixed together to produce an equimolar blend. This resin blend was converted into molding compound by mixing it with 418.0 g of milled carbon fiber and 119.5 g of graphite powder. The wet mixture was first dried in a vacuum oven at about 70C for 1 ¼ hr and then in an air-circulating oven at 102C for 3 hr more. The dried molding compound was granulated and then molded into a plaque measuring 9 cm×13 cm 0.66 cm, with a density of 1.40 g/cc. Flexural test bars were machined from the plaque and tested. Flexural strength averaged 84.3 MPa for the as-molded test bars. Flexural modulus was 15.5 GPa.

### Example 12

Dried Benzoxazine Resin with MDA (n=1) Co-Cured with BAPP, an Aromatic Primary Diamine

A portion of the monomer solution described in Example 7 was first dried in a vacuum oven at about 70C for 1 ½ hr and then in an air-circulating oven at 102C for 3 hr more. The dried “cake” (55.6 g=0.059 mole) was broken into small chunks and ballmilled into a fine, pale yellow powder. A roughly equimolar quantity of BAPP (22.4 g=0.055 mole) was added to the ballmill and the two powders were blended. The powder blend was molded into a plaque measuring 9 cm×13 cm×0.60 cm that had a density of 1.17 g/cc and was translucent cherry-red in backlighting.

### Example 13

Benzoxazine Resin with PPDA (n=2) Prepared in Solvent Dried, and Co-Cured with a Standard DGEBA Epoxy Resin

A 3000-mL glass kettle was placed in an ice bath to keep it cool. 350 g of toluene and 200 g of ethyl acetate were added, followed by 148.2 g of formalin (1.83 moles), 114.4 g of aniline (1.23 mole,), 174.6 g of paraformaldehyde (5.53 moles), 131.3 g of p-phenylenediamine, and 416.4 g of bisphenol A (1.83 moles). The mixture was slowly heated to reflux at about 73 C. Reflux was maintained for about 4 ¾ hr, during which time 101 g of aqueous phase was removed (out of calculated 257 g total). The final resin solution was light brown in color, opaque, tacky but pourable.

The resin solution was dried in an air-circulating oven for 6 hr at 213 C. The dried “cake” was broken into small chunks, ballmilled for 12 hr into a light yellow powder, which was then sifted through a 30-mesh screen to remove oversize particles. A molding compound was made by mixing by hand 45 g of the MBZ powder, 45 g of Dow DER-331 epoxy resin, and 10 g of graphite powder. The mixture had the consistency of peanut butter. In the mold, it liquefied at 96C, at which point it was degassed under vacuum. Molding was completed at about 205C. The molded plaque was black and measured 9cm×13cm×0.69cm.

### Example 14

Benzoxazine Resin with Bisphenol F and MDA (n=1) Prepared in Solvent: F.M.W.=896 g/mole

A 3000-mL glass kettle was placed in an ice bath to keep it cool. 700 g of toluene and 100 g of ethyl acetate were added, followed by 174.1 g of formalin (2.15 moles), 249.1 g of aniline (2.68 moles), 270.7 g of paraformaldehyde (8.57 moles), 265.2 g of 4,4′-methylenedianiline (1.38 moles), and 557.2 g of bisphenol F (2.62 moles). The mixture was slowly heated to reflux at 82C. Reflux was maintained for about 2 ¾ hr, during which time 144 g of aqueous phase was removed (out of calculated 295 g total). The final resin solution was light-lemon in color, snotty and very viscous.

The resin solution was converted into molding compound by mixing a third of it with 252.1 g of milled carbon fiber and 72.2 g of graphite powder. The wet mixture was dried in an air-circulating oven at 113C for 3 hr. The dried molding compound was granulated and then molded into a plaque measuring 28 cm×41 cm×0.65 cm, with a density of 1.43 g/cc. Flexural test bars were machined from the plaque and tested. Flexural strength averaged 77.2 MPa for as-molded test bars. Flexural modulus was 17.3 GPa.

### Example 15

Benzoxazine Resin with Phenol Endcaps and MDA (n=1) Prepared in Solvent; F.M.W.=909 g/mole

A 1000-ml glass kettle was placed in an ice bath to deep it cool. 200 g of toluene and 30 g of ethyl acetate were added, followed by 54.1 g of formalin (0.667 mole), 63.2 g of paraformaldehyde (2.00 moles), 62.7 g of phenol (0.667 mole), 76.2 g of bisphenol A (0.333 mole) and 132.2 g of 4,4′-methylenedianiline (0.667 mole). The mixture was slowly heated to reflux at 83C. Reflux was maintained for 70 minutes, during which time 27.5 g of aqueous phase was removed (out of calculated 53 g total). The final solution was off-white in color and opaque. It gelled while cooling but the nontacky gelled material was easily cut and handled.

The gelatinous resin was sliced and placed in aluminum pans for drying. It was first dried in a vacuum oven at about 70C for a half hour and then in an air-circulating oven at 104C for 2 hours more. The dried material was granulated and molded into a plaque measuring 9 cm×13 cm×0.66 cm, with a density of 1.176 g/cc. The molded plaque was dark red in color and translucent to backlighting.

The invention has been described in detail, with particular emphasis having been placed on the preferred embodiments, but variations and modifications within the spirit and scope of the invention may occur to those skilled in the art to which it pertains.

What is claimed is:

1. A benzoxazine monomer, comprising:
at least one aromatic primary monoamine to serve as an endcap, at least one polyphenol, at least one aldehyde, and at least one aromatic primary polyamine.

2. The benzoxazine monomer of claim 1 wherein the endcap is a monoamine, the polyphenol is a diphenol, and the polyamine is an aromatic primary diamine, having the idealized structure represented as
A-(B-D)ₙ-B-A.

3. The monomer of claim 2 wherein each linkage is an oxazine ring formed by the consumption of two aldehyde molecules.

4. The monomer of claim 2 wherein subscript “n” is a number from above zero to about ten.

5. The monomer of claim 4 wherein subscript “n” is in the range of at least 0.1 to 3.

6. The monomer of claim 1 wherein the aromatic primary monoamine endcap is selected from the group consisting of aniline, toluidene, xylidene, and napthylamine.

7. The monomer of claim 1 wherein the polyphenol is a diphenol and is selected from the group consisting of resorcinol, bisphenol A, bisphenol F, bisphenol S, biphenol, and dihydroxybenzophenone.

8. The monomer of claim 1 wherein the polyphenol is a triphenol.

9. The monomer of claim 8 wherein the triphenol is trisphenol-PA.

10. The monomer of claim 1 wherein the aldehyde is formaldehyde and is selected from the group consisting of paraformaldehyde, trioxane, polyoxymethylene, and formalin.

11. The monomer of claim 1 wherein the aromatic primary amine chain extender is selected from the group consisting of phenylenediamine, methylenedianiline, oxydianiline, diaminodiphenylsulfone, 2,2-bis(4-[aminophenoxy]phenyl)propane, oxydianiline, and diaminobenzanilide.

12. A benzoxazine monomer, comprising phenol or at least one substituted phenol to serve as endcap, at least one polyphenol, at least one aromatic primary polyamine and at least one aldehyde.

13. The benzoxazine monomer of claim 12 wherein the endcap is phenol or substituted phenol, the polyphenol is a diphenol, and the polyamine is an aromatic primary diamine, having the idealized structure represented as
P-(D-B)ₙ-D-P

14. The monomer of claim 13 wherein each linkage is an oxazine ring formed by the consumption of two aldehyde molecules.

15. The monomer of claim 13 wherein subscript “n” is a number from above zero to about ten.

16. The monomer of claim 15 wherein subscript “n” is in the range of a least 0.1 to 3.

17. A method of preparing a benzoxazine monomer in a solvent, the reactants including at least one aromatic primary monoamine, at least one polyphenol, at least one aldehyde, and at least one aromatic primary diamine, comprising combining said reactants at a temperature in the range of 0° C. to 25° C. in a reaction kettle to form a resinous solution, heating the kettle to reflux, and holding the kettle at that temperature until at least a third of the water has been removed by azeotropic distillation, the resinous solution being dried in vacuo, and spreading the friable solid in flat pans and further drying the friable solid at atmospheric pressure at or slightly above its melting point, and holding the friable solid at that temperature for at least 10 minutes in order to complete the process.

18. The method of claim 17 wherein the reactants include formaldehyde selected from the group consisting of paraformaldehyde, trioxane, polyoxymethylene, and formalin.

19. The method of claim 18 wherein the formaldehyde is a mixture of paraformaldehyde and formalin in a ratio from 10:1 to 1:4, based on the dry weight of formaldehyde.

20. The method of claim 19 wherein the ratio is in the range of 5:1 to 1:1.

21. The method of claim 17 wherein the solvent comprises a mixture of toluene and ethyl acetate in the ratio of 10:1 to 1:4.

22. The method of claim 21 wherein the ratio is in the range of 8:1 to 6:1.

23. The method of claim 17 wherein the aromatic primary monoamine is selected from the group consisting of aniline, toluidene, xylidene, and napthylamine.

24. The method of claim 17 wherein the polyphenol is a diphenol and is selected from the group consisting of resorcinol, bisphenol A, bisphenol F, bisphenol S, biphenol, and dihydroxybenzophenone.

25. The method of claim 17 wherein the aromatic primary amine chain extender is selected from the group consisting of phenylenediamine, methylenedianiline, oxydianiline, diaminodiphenylsulfone, 2,2-bis(4-[aminophenoxy]phenyl)propane, 4,4′-oxydianiline, and diaminobenzanilide.

26. The method of claim 17 and including the step of blending the resinous solution with particulate fillers by mechanical mixing to form a molding compound; drying the compound to remove excess solvent and water; heating the dried compound to a temperature in the range of 100° C. to 130° C. so as to cause melting; holding the compound at the melt temperature for 10 to 60 minutes; and subsequently granulating the compound.

27. The method of claim 26 wherein the resinous solution can optionally be applied to reinforcing fibers in order form a towpreg or prepreg.

28. The method of claim 26 wherein the compound is dried in vacuo.

29. The method of claim 26 wherein the compound is dried in vacuo at a temperature in the range of 0° C. to 100° C.

30. A method of preparing a benzoxazine monomer from reactants including at least one aromatic primary monoamine, at least one polyphenol, at least one aldehyde, and at least one aromatic primary diamine in an aqueous slurry comprising the steps of combining the reactants with a minimal amount of water at a temperature from about 0° C. to about 25° C. in a reaction kettle; and ultimately grinding the solid product into a powder.

31. The method of claim 30 and including the step of combining the reactants and water at a temperature in the range of 0° C. to 10° C.

32. The method of claim 30 wherein the formaldehyde is preferably in the form of formalin.

33. The method of claim 32 wherein the formalin is about 37% by weight of formaldehyde in water or in water/methanol.

34. The method of claim 30 wherein the liquid medium comprises water and methanol, and said water comprising at least 20% of the weight of the mixture.

35. A benzoxazine homopolymer suited for applications in the temperature range of about 170° F. to 260° C., said polymer being made from benzoxazine monomer cured in the temperature range of about 170° F. to 260° C. and cured under applied pressure.

36. A polymer suitable for applications in the range of 170° C. to 260° C. wherein the benzoxazine monomer is co-cured with a primary or secondary polyamine having an equivalent thermal stability and selected from the group comprising phenylenediamine, methylenedianiline, oxydianiline, diaminodiphenylsulfone, 2,2-bis(4-[aminophenoxy]phenyl)propane, and diaminobenzanilide.

37. A polymer suitable for applications in the range of 170° C. to 260° C. wherein the benzoxazine monomer is co-cured with an epoxy resin.

38. A polymer suitable for applications in the range of 170° C. to 260° C. wherein the benzoxazine monomer is co-cured with a phenolic resin.
